(19) United States
(12) Patent Application Publication
(10) Pub. No.: US 2014/0045804 A1
(43) Pub. Date: Feb. 13, 2014

(54) TREATMENT OF HYPOACTIVE SEXUAL DISORDER (HSDD)

(71) Applicant: Edward M. Lichten, Birmingham, MI (US)

(72) Inventor: Edward M. Lichten, Birmingham, MI (US)

(21) Appl. No.: 13/745,934

(22) Filed: Jan. 21, 2013

Related U.S. Application Data
(60) Provisional application No. 61/588,248, filed on Jan. 19, 2012.

(51) Int. Cl. A61K 31/58

(52) U.S. Cl. CPC A61K 31/58
USPC 514/176; 540/52

(57) ABSTRACT

A topical cream to be applied to the clitoris and hood of the vaginal area to create an intense libido and drive for orgasm. The cream comprises a base cream having from about 9.5% to about 10%, by volume, of a steroid, based upon the total volume of the cream. Preferably, at least about 1% to about 10% by volume, of the steroid, based on the total volume of the cream. The topical cream may, also, include from about 0.1% to about 2.0%, by volume, of oxytocin. The addition of the small amount thereof enhances the efficacy of the cream.

### CROSS REFERENCE TO RELATED APPLICATION

This application claims the benefit of U.S. Provisional Application 61/588,248, which was filed on Jan. 19, 2012, the disclosure of which is hereby incorporated by reference.

### BACKGROUND OF THE INVENTION

The physiology of orgasm is incomplete. It has been reported that greater than about 27% to about 33% of menopausal women have decreased desire for sex and that 25%-33.5% of menopausal women report being unable to achieve orgasm. Although physical symptoms of stress, fatigue, lack of interest in the partner and dissatisfaction with physical appearance are often quoted as the underlying causes for the failure to achieve orgasm, many physicians classify these women with clusters of psychological and social problems.

Heretofore, little attention has been directed to addressing local physiological effect that stimulates vagal nerves, which may serve as a neuropeptide mediator to facilitate orgasm in women.

It has been noted that oxytocin has both local and neuropeptide effect in the electric transmission of afferent nerve impulses to the limbic brain centers, but with little reported success.

Thus, the present invention, as described hereinafter, is directed to causing a local physiological effect that stimulates the vagal nerves to mediate the neuropeptides.

### DESCRIPTION OF THE PREFERRED EMBODIMENT

Thus, in accordance herewith there is provided a topical cream which, when applied to the clitoris and hood of the vaginal area, creates an intense libido and drive for orgasm.

The cream, generally, comprises a base cream having from about 9.5% to about 10%, by volume, of a steroid, based upon the total volume of the cream; and preferably, at least about 1% to about 10% by volume, of the steroid, based on the total volume of the cream.

The topical cream may, also, include from about 0.1% to about 2.0%, by volume, of oxytocin. The addition of the small amount thereof enhances the efficacy of the cream.

Suitable base creams, as is known to the skilled artisan, includes emollients, water, alcohols, such as isopropyl alcohol and benzyl alcohol, glycerol, fragrances, moisturizers and the like as well as mixtures thereof.

A particularly preferred base cream is that sold under the mark Lipoderm. Lipoderm is reported as having two forms, Lipoderm Ultra and Lipoderm Y, each, comprising a proprietary blend of yohimbine HCl, caffeine, synephrine, acetyl L-carntine, lechithin, sesamin, octopamine HCl and ascorbyl palmitate.

It should be noted that the base cream may be used alone or in admixture with other creams such as commercially available face creams, body moisturizing creams and the like, as well as mixtures thereof.

Where used the base cream and other additional cream may be used in any amount up to about a 50:50 volumetric mixture thereof of base cream to additional cream.

Among the useful steroids are certain androgens including stanozolol, testosterone and the like. Preferably, the steroid is stanozolol. The stanozolol may be used alone or in admixture with other steroids including testosterone.

Other useful androgens include, for example, dehydroepiandrosterone, androstenedione, androstenediol, androsterone, dihydrotestosterone, nandrolone, oxandrin, furzabolin and the like may be used, as well as mixtures thereof. It is possible, although not preferred, to use a combination of synthetic and natural androgens including anabolic steroids. These are well known and commercially available.

Preferably, the stanozolol is used alone. However, when an additional steroid is used, testosterone is the preferred secondary steroid. Where the secondary steroid is used, it is used in minor amounts in a primary to secondary steroid volumetric ratio of about 95:5.

The cream is prepared by admixing, at room temperature the base cream and the stanozolol, and the other ingredients, where used, are stirred to form a homogenous composition.

Although not wishing to be bound by any theory, it is believed that by manual stimulation of the clitoris, the pudendal nerve is sensitized.

Also, it is hypothesized that there is a local physiological effect whereby stanozolol releases testosterone which, in turn, releases oxytocin and, thus, serves as a neuropeptide mediator. Concomitantly the stanozolol minimizes the effect of the sex hormone binding globulin.

For a more complete understanding of the present invention reference is made to the following illustrated Example. In the Example all parts are by volume, absent indications to the contrary.

### EXAMPLE

A topical cream is prepared by mixing together, at room temperature, 60 ml of Lipoderm Ultra, to which is added about 1.2 grams of stanozolol and 10 iUs of oxytocin.

Thereafter, ten menopausal women with a history of a decreased sexual response (HSDD) or lack of orgasm are used to test the efficacy of the cream.

Each woman had prescreening laboratory tests. Where possible, hormonal, electrolyte and mineral replacement is initiated without replacement of ovarian and adrenal hormones, e.g. estrogen, progesterone, testosterone, and DHEA.

The women are situated comfortably in a lounge chair, and a base line blood sample is drawn. Each woman is instructed to apply 1 ml of cream to the vaginal hood and clitoris. Repeated blood drawing is then drawn at 15, 30, 45 and 60 minutes after application.

Each of the women are asked to rate their response to the cream first on libido without stimulation and then with a second application with stimulation.

The results show that in the women, void of any hormonal replacement a 1 ml application of the cream applied to a woman's clitoris and hood creates intense libido and drive for orgasm with or without the presence of the partner.

Measurements of serum assays of testosterone, estradiol, progesterone, oxytocin, prolactin and sex hormone binding globin show no significant changes. This implies that the effect of the cream is limited to local action, and that resultant neuro-stimulation may trigger or bypass the expected spinal pathways.

It has been found also, there is a spinothalamic ganglion or plexus, which when stimulated along with clitoral stimulation, can result in orgasm in a paraplegic woman.

Further, it has been found that the present cream increases sexual drive if applied to other female sensory areas such as the breast nipples.

From the above, it is to be appreciated that there is described herein a topical cream which has a positive effect on women having hypoactive sexual disorder.

I claim:

1. A topical cream for enhancing sexual activity comprising a steroid, the steroid being androgen.

2. The topical cream of claim 1 wherein the androgren is stanozolal.

3. The topical cream of claim 2 wherein the steroid comprises about 1% to about 10% by volume, based on the total volume of the cream.
